(19) United States
(12) Patent Application Publication
(10) Pub. No.: US 2010/0160940 A1
(43) Pub. Date: Jun. 24, 2010

(54) Surgical Instrument

(75) Inventors: Theodor Lutze, Balgheim (DE); Dirk Schauer, Berlin (DE); Alexander Disch, Freiburg (DE); Holger Reinecke, Emmendingen (DE); Claas Mueller, Freiburg (DE)

Correspondence Address: Lipsitz & McAllister, LLC, 755 MAIN STREET, MONROE, CT, 06468, US

(73) Assignees: Aesculap AG, Tuttlingen (DE); Albert-Ludwigs-Universitaet Freiburg, Freiburg (DE)

(21) Appl. No.: 12/653,733

(22) Filed: Dec. 17, 2009

Related U.S. Application Data
(63) Continuation of application No. PCT/EP2008/058205, filed on Jun. 26, 2008.

(30) Foreign Application Priority Data
Jun. 26, 2007 (DE) 10 2007 030 854

(51) Int. Cl. A61B 17/28; A61B 17/32

(52) U.S. Cl. 606/170; 606/205

(57) ABSTRACT

A surgical instrument has a shaft, an end effector comprising at least one movably mounted tool element and defining a longitudinal axis, and a force transmitting member mounted in the shaft for movement in the distal and proximal directions for transmitting an actuating force introduced at a proximal end of the instrument onto the end effector in order to move the at least one tool element from a first tool element position to a second tool element position and/or vice versa. The at least one tool element is pivotably mounted on a bearing shaft held on the shaft. The at least one tool element is connected by at least one articulation member, at least a section of which is flexible, to the force transmitting member in order to transmit the actuating force from the force transmitting member onto the at least one tool element.

This application is a continuation of international application number PCT/EP2008/058205 filed on Jun. 26, 2008.

The present disclosure relates to the subject matter disclosed in international application number PCT/EP2008/058205 of Jun. 26, 2008 and German application number 10 2007 030854.1 of Jun. 26, 2007, which are incorporated herein by reference in their entirety and for all purposes.

### FIELD OF THE INVENTION

The present invention relates to surgical instruments generally, and more specifically to a surgical instrument in the form of tubular shaft instruments in minimally invasive surgery.

### BACKGROUND OF THE INVENTION

The present invention relates to a surgical instrument having a shaft, an end effector comprising at least one movably mounted tool element and defining a longitudinal axis, and a force transmitting member mounted in the shaft for movement in the distal and proximal directions for transmitting an actuating force introduced at a proximal end of the instrument onto the end effector in order to move the at least one tool element from a first tool element position to a second tool element position and/or vice versa, the at least one tool element being pivotably mounted on a bearing shaft held on the shaft.

Surgical instruments of the kind described at the outset are used in numerous variants in the form of so-called tubular shaft instruments in minimally invasive surgery. It is known to connect the pivotably mounted tool elements by means of links pivotably mounted on the tool element and on the force transmitting member and to transmit actuating forces in order to move the tool elements. A further alternative is the provision of so-called “slot-cam-guides”, with on either the tool element or the force transmitting member a recess defining a guide track and on the respective other part a projection engaging the guide track in order to move the projection in the guide track as a result of a relative movement of the force transmitting member and the shaft and thereby bring about a movement of the at least one tool element.

However, in all known articulations of the instruments available on the market, a play occurring as a result of unavoidable manufacturing tolerances and frictional losses occurring on account of the articulation prove to be disadvantageous. In particular, these disadvantages result in an inability to miniaturize the known instruments in a desired manner while maintaining their stability and functionality. Shaft diameters which are significantly smaller than 10 mm are not achievable in practice with conventional force transmitting mechanisms.

### SUMMARY OF THE INVENTION

In accordance with the present invention, a surgical instrument is provided with an end effector which can be constructed significantly smaller than in conventional instruments.

In a first aspect of the invention, a surgical instrument having a shaft, an end effector comprising at least one movably mounted tool element and defining a longitudinal axis, and a force transmitting member mounted in the shaft for, movement in the distal and proximal directions for transmitting an actuating force introduced at a proximal end of the instrument onto the end effector in order to move the at least one tool element from a first tool element position to a second tool element position and/or vice versa, the at least one tool element being pivotably mounted on a bearing shaft held on the shaft. The at least one tool element is connected by at least one articulation member, at least a section of which is flexible, to the force transmitting member in order to transmit the actuating force from the force transmitting member onto the at least one tool element.

### BRIEF DESCRIPTION OF THE DRAWINGS

The foregoing summary and the following description may be better understood in conjunction with the drawing figures, of which:

FIG. 1 shows an overall perspective view, partially broken open, of a surgical instrument:

FIG. 2 shows a perspective enlarged view of area A from FIG. 1;

FIG. 3 shows a perspective representation of an end effector of the instrument shown in FIGS. 1 and 2 in a connected position;

FIG. 4 shows an exploded representation of the end effector from FIG. 3;

FIG. 5 shows a side view, partially broken open, of an alternative embodiment of a surgical instrument;

FIG. 6 shows a perspective view of a further embodiment of an end effector;

FIG. 7 shows a longitudinal sectional view of the end effector from FIG. 6;

FIG. 8 shows an exploded representation of the end effector from FIG. 6 seen from above;

FIG. 9 shows an exploded representation of the end effector from FIG. 6 seen from below;

FIG. 10 shows a perspective view of a further end effector constructed in the form of a cutting tool;

FIG. 11 shows an exploded representation of the end effector from FIG. 10 seen from above;

FIG. 12 shows an exploded representation of the end effector from FIG. 10 seen from below;

FIG. 13 shows a perspective view of a further end effector, constructed in the form of a gripper, in a normal position;

FIG. 14 shows a side view of the end effector shown in FIG. 13;

FIG. 15 shows an exploded representation of the end effector shown in FIG. 13; and

FIG. 16 shows a side view of the end effector shown in FIG. 13 in a pulled position with supported articulation members.

### DETAILED DESCRIPTION OF PREFERRED EMBODIMENTS OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a surgical instrument having a shaft, an end effector comprising at least one movably mounted tool element and defining a longitudinal axis, and a force transmitting member mounted in the shaft for movement in the distal and proximal directions for transmitting an actuating force introduced at a proximal end of the instrument onto the end effector in order to move the at least one tool element from a first tool element position to a second tool element position and/or vice versa, the at least one tool element being pivotably mounted on a bearing shaft held on the shaft, the at least one tool element is connected by at least one articulation member, at least a section of which is flexible, to the force transmitting member in order to transmit the actuating force from the force transmitting member onto the at least one tool element.

The articulation member which, of course, may also be of fully flexible construction, i.e., over its entire length, permits a deformation which is required for a movement of the at least one tool element, so that known articulation variants such as, for example, “slot-cam-guides” or pivotable articulations of small links between the at least one tool element and the force transmitting member can be dispensed with. Therefore, in comparison with the prior art solutions, the frictional losses are minimized by provision of at least one articulation member in the described manner. In addition, the articulation, proposed in accordance with the invention, of the at least one tool element has extremely little play, and, depending on the configuration, is even practically free of play. Since complicated articulations between the force transmitting member and the at least one tool element can be dispensed with, such an articulation member makes it possible to significantly reduce a size of the end effector, i.e., in particular, to also produce diameters of end effectors which may be smaller than 2 mm. The instrument proposed is, therefore, well-suited for use, in particular, in neurosurgical procedures or minimally invasive operations on children.

The end effector has particularly little play and friction when the at least one articulation member is undetachably connected to the at least one tool element. Furthermore, parts of particularly small end effectors can thereby be prevented from becoming detached and getting lost in a patient's body.

A surgical instrument can be manufactured with particular ease and provided with sufficient stability when the at least one articulation member is formed in one piece with the at least one tool element.

Preferably, the at least one articulation member is undetachably connected to the force transmitting member. Frictional losses, also in the area of the force transmission from the force transmitting member onto the articulation member, can thereby be minimized and the articulation constructed in a particularly play-free manner.

The stability of the end effector can be further increased by the at least one articulation member being formed in one piece with the force transmitting member.

In particular, it is advantageous for the at least one articulation member to form a flexure hinge between the force transmitting member and the at least one tool element. By providing a flexure hinge, all other forms of engaging parts can be dispensed with for transmitting a force from the force transmitting member onto the at least one tool element. Therefore, flexure hinges enable a practically play-free force transmission and even in the case of maximum miniaturization still have sufficient stability for any desired function of the instrument.

To enable pivoting of the at least one tool element away from the longitudinal axis, in particular, by transmitting a pulling force, it is expedient for the at least one articulation member to engage the at least one tool element on an outer side facing away from the longitudinal axis.

It may, however, also be expedient for the at least one articulation member to engage the at least one tool element on an inner side facing the longitudinal axis or on an inner section. This configuration makes it possible to move the at least one tool element in the direction towards the longitudinal axis, in particular, by acting upon the articulation member with a pulling force. Therefore, where two tool elements are provided, these can be closed as a result of a pulling force exerted thereon.

An overall stability of the instrument can be further increased by a proximal end of the end effector being fixedly connected to the force transmitting member.

Preferably, a proximal end of the end effector is formed in one piece with the force transmitting member. In the manufacture, it is therefore possible to dispense with a connection between the end effector and the force transmitting member, which may be complicated and, in addition, may limit a stability of the instrument.

In accordance with a preferred embodiment, it may be provided that the end effector comprises a force transmitting section movable in the distal and proximal directions, which is connected to or lies against the force transmitting member and forms a distal end area of the force transmitting member, and that a proximal end of the at least one articulation member is connected to the force transmitting section. By providing the force transmitting section, the end effector can be formed as an independent unit, in particular, also in one piece. This has manufacturing advantages, in particular, when the end effector is to be highly miniaturized. The force transmitting member and the end effector can therefore be manufactured separately. The force transmission onto the at least one tool element then occurs from the force transmitting section through the articulation member onto the tool element to be moved.

A particularly compact construction of the end effector is achieved, in particular, by the at least one articulation member being formed in one piece with the force transmitting section.

In particular, one of the above-described end effectors can be used in a simple way in conjunction with conventional instruments when the force transmitting member comprises a push-and-pull rod. In particular, the force transmitting member itself may be constructed as a push-and-pull rod.

Furthermore, it is advantageous for the force transmitting member to comprise at least one piston mounted for displacement in the shaft, and a fluid section separated from the at least one piston by a sealing element and filled with a fluid which is pressable against the sealing element by the application of pressure in order to move the at least one piston lying against the sealing element parallel to the longitudinal axis of the shaft. Provision of such a fluid section makes it possible for a shaft to be made of flexible or curved configuration and to nevertheless ensure a desired force transmission from a proximal end of the instrument to the at least one tool element. In addition, a fluidic force transmission has particularly little play and, as a rule, is even practically free of play, and has almost no frictional losses.

The instrument can be of particularly compact construction when the at least one piston comprises the force transmitting section. In particular, the piston can form the force transmitting section.

To form a closed fluidic force transmission, it is expedient for the force transmitting member to comprise two pistons which delimit between them distally and proximally the fluid section separated from the pistons by a respective sealing element.

To prevent losses of a fluid contained in the fluid section, which is preferably a fluid that is compatible with the body, for example, a physiological saline solution, it is expedient for the fluid section to comprise a fluid space which is completely closed and sealed off.

In particular, optimum sealing of the fluid section by the sealing element can be achieved by the sealing element being flexible and inelastic. A cover of the fluid section is thereby prevented from bursting as a result of an increase of pressure in the fluid.

When a sealing element is in the form of a bellows or a rolling bellows it is then easy to manufacture and easy to connect to a shaft and, in addition, it permits long displacement paths.

To prevent rotation of the force transmitting section and/or the force transmitting member relative to the shaft about its longitudinal axis, it is advantageous for the instrument to comprise a guide device for guiding a movement of the force transmitting section and/or the force transmitting member in the shaft parallel to the longitudinal axis.

Expediently, the guide device forms a locking device against rotation in order to prevent rotation of the at least one piston about the longitudinal axis. In particular, a jamming of the end effector in the shaft or of the at least one tool element on the bearing shaft can thereby be reliably prevented.

The construction of the guide device is particularly simple when it comprises first and second interacting guide members, one of which is arranged on the force transmitting section and the other on the shaft, and when the first guide member is in the form of a guide projection and the second guide member is in the form of a guide recess.

A longitudinal guide can be formed in a simple way by the guide recess being in the form of a guide groove extending parallel to the longitudinal axis. In particular, a corresponding guide projection can move in this guide groove parallel to the longitudinal axis.

To enable easy and reliable movement of the force transmitting member parallel to the longitudinal axis, preferably in the distal and proximal directions, for an operator, an actuating device is expediently provided at the proximal end of the instrument for moving the force transmitting member parallel to the longitudinal axis.

Handling of the instrument is preferably improved by the actuating device comprising at least one movable grip element which is articulatedly connected to the force transmitting member.

In particular, it is also to be understood by an articulated connection that the grip element lies against a proximal end of the force transmitting member and is able to move it in the distal direction.

To enable easy cleaning of the instrument, it is advantageous for the actuating device to be detachably connectable to the shaft and/or the force transmitting member. In particular, it is also conceivable to construct the shaft with the end effector as a unit which can be used as a one-way product for use once with a reusable actuating device.

To enable a surgeon to recognize an orientation of the end effector, in particular, of the at least one tool element, directly at a position of the actuating device, it is expedient for the shaft to be arranged stationarily relative to the actuating device.

To enable a surgeon to hold the instrument comfortably in a convenient position and yet adjust the at least one tool element in the necessary way inside a patient's body for performance of a surgical operation, it is advantageous for the shaft and/or the force transmitting member to be rotatable relative to the actuating device about the longitudinal axis.

It is expedient for the at least one articulation member to be undeformed in a position of the tool element defining a normal position. In particular, an instrument with two tool elements can be constructed such that the two tool elements are closed or spread open in the normal position. Accordingly, in the normal position the at least one articulation member is unable to exert any forces on the at least one tool element or the force transmitting member.

In accordance with a further preferred embodiment, it may be provided that the at least one articulation member is deformed in any operating position of the at least one tool element that differs from a normal position in which it is undeformed. The operating position is also a position of the tool element, but it does not coincide with the normal position. This construction of the instrument has the advantage that deformation of the articulation member enables energy to be stored for transferring the at least one tool element fully or partially from the swivelled operating position back into the normal position again. The articulation member therefore simultaneously serves as resetting member of a resetting device.

To construct the instrument, in particular, its end effector, so that it is as slim and compact as possible, it is expedient for the at least one articulation member to extend parallel or substantially parallel to the longitudinal axis both proximally and distally of the bearing shaft. Furthermore, a desired opening angle or pivot angle of the at least one tool element can also be achieved by a corresponding length of the articulation member. A pivotal movement of the at least one tool element can also be optionally set by a corresponding delimitation at the actuating device or by a corresponding delimitation of a displacement path of the force transmitting member in the shaft.

The end effector can be of particularly compact construction when the at least one articulation member extends equally or approximately equally far proximally and distally of the bearing shaft. In particular, all of the parts required for the articulation of the at least one tool element can therefore be arranged one behind the other in the direction of the longitudinal axis, thereby significantly reducing an outer diameter of the shaft and an outer diameter of the end effector, respectively.

The construction of the end effector is particularly simple when the at least one articulation member is in the form of a thin web. In particular, a thickness of the preferably strip-shaped web can be a great deal smaller than a width thereof. Ratios of 1:10 or even smaller than 1:20 are possible.

To construct a particularly small end effector, it is expedient for the articulation member, particularly when it is in the form of a web, to have a thickness ranging from approximately 50 μm to 700 μm. The thickness is preferably from 100 μm to 200 μm.

In order that the at least one tool element, which is swivelled out of a normal position, can be automatically transferred back into it when no actuating force is exerted on the force transmitting member, it is advantageous for a resetting device to be provided for transferring the at least one tool element from any swivelled position of the tool element back into a normal position.

The construction of the resetting device is particularly simple when it comprises at least one resetting member which is supported, on the one hand, on the at least one tool element, and, on the other hand, on the force transmitting member. In particular, the resetting member can also be supported on the force transmitting section of the end effector if, for example, this forms a distal end of the force transmitting member.

A resetting force can be applied, in particular, in a simple way by the at least one resetting member when it is resiliently elastic. Energy stored as a result of deformation of the resetting member can then be used, in turn, to transfer the at least one tool element from a swivelled position of the tool element back into the normal position.

The construction of the instrument, in particular, of the end effector, can be made particularly compact when the at least one articulation member forms the resetting member. The articulation member therefore has a double function. In this case, a separate resetting member can be completely dispensed with.

The construction of the instrument is simplified and therefore, in particular, also its manufacture when the distal end of the instrument is of mirror-symmetrical configuration in relation to at least one plane of symmetry. Therefore, in particular when two or more tool elements are provided, a symmetrical actuation of these is then already prescribed by the construction of the end effector.

The end effector is particularly simply constructed and easily manufactured when the force transmitting section is of two-part construction and comprises two section halves essentially separated by a plane containing the longitudinal axis. For example, a force transmitting section of overall substantially cylindrical shape can be formed by two cylinder halves.

The instrument can be constructed in a simple way in the form of scissors, tweezers or grasping forceps when it comprises two tool elements.

The manufacture of the end effector can be simplified, in particular, by it being of two-part construction and comprising two end effector parts. In particular, complicated shapes can then be formed more easily on the tool elements, for example, by milling, wire or laser erosion.

To construct a symmetrical or substantially symmetrical end effector, it is expedient for each end effector part to comprise a tool element.

Furthermore, the manufacture can be simplified and the stability of each end effector part increased by each end effector part comprising one section half of the force transmitting section.

In order that only one kind or type of end effector part need be manufactured when constructing a two-part end effector, it is expedient for the end effector to be formed by two identical end effector parts fixedly connected to each other, which are arranged in a manner rotated through 180° about the longitudinal axis relative to each other. Two identical effector parts can then be fitted together to form a single end effector.

In accordance with a further preferred embodiment, it may be provided that the two tool elements are constructed so as to mutually penetrate or intersect each other. In particular, it is to be understood by this that there can be provided on one tool element an opening or recess through which the other tool element, for example, a distal end thereof, is guided or introduced. A penetrating or intersecting construction of the tool elements enables, in particular, a pivotal movement thereof towards each other by a pulling force acting on the articulation member. In particular, scissors, grasping forceps and tweezers, which are open in a normal position and can be closed by exerting a pulling force on the force transmitting member, can be constructed in this way.

Preferably, a connecting device is provided for connecting the end effector parts to each other. The end effector parts can therefore be manufactured separately, connected to each other and then inserted in the shaft of the instrument. Optionally, they may, of course, also be adhesively connected or welded to each other.

The construction of the end effector and therefore also of the instrument is particularly simple when the connecting device comprises at least one first and at least one second connecting element, which are in engagement with each other in a connected position, and when the at least one first and the at least one second connecting element are each arranged on one end effector part. Two end effector parts can therefore be connected to each other in a simple way by the at least one first and second connecting elements being brought into engagement with each other so that they assume the connected position.

The construction of the connecting device is particularly simple when the at least one first connecting element is in the form of a projection, and the at least one second connecting element is in the form of a receptacle corresponding to the projection. Projections and openings can be made particularly easily. In addition, they may also serve as mutual stops for prescribing in a desired manner a relative position of the end effector parts that are to be connected to each other.

Preferably, the at least one first and the at least one second connecting element are oriented so as to face in a direction transverse or substantially transverse to the longitudinal axis. For example, they can be constructed so as to project from one end effector part and face the other end effector part, more specifically, transversely to the longitudinal axis. This prevents the end effector parts from being able to be moved apart as a result of a movement of the force transmitting member parallel to the longitudinal axis.

A connection of two end effector parts is particularly simple when the at least one first and the at least one second connecting element extend in a direction parallel or substantially parallel to the longitudinal axis. For example, the connecting elements may be in the form of grooves and projections engaging these with a positive connection, with the projections facing away from a respective end effector part transversely to the longitudinal axis, but extending in a direction parallel to the longitudinal axis. Alternatively, it is also conceivable for a groove to extend transversely to the longitudinal axis and to form a connecting element. In particular, the connecting elements engaging with one another may be constructed for engagement in a clamping and/or latching manner with one another in the connected position. Optionally, an, in particular, also limited, relative movability of the two end effector parts in the connected position may be desirable for adjustment purposes.

To enable particularly compact construction of the end effector, it is expedient for the connecting device to be arranged on the force transmitting section for connecting the section halves. The connecting device does therefore not interfere spatially with the at least one tool element, in particular, with movement thereof.

In accordance with a preferred embodiment, it may be provided that the end effector is so constructed that as a result of a movement of the force transmitting member in the proximal direction, the at least one tool element is pivotable towards the longitudinal axis and vice versa. With such an end effector, grasping forceps, for example, can be formed, which close when a pulling force is applied.

Furthermore, it is advantageous for the end effector to be so constructed that as a result of a movement of the force transmitting member in the distal direction, the at least one tool element is pivotable towards the longitudinal axis and vice versa. For example, a surgical instrument such as scissors or grasping forceps can be constructed in this way, which can be closed as a result of pressure acting in the distal direction being applied to the force transmitting member.

Furthermore, it may be expedient for the end effector to comprise two tool elements and to be so constructed that as a result of a pushing force transmittable by the force transmitting member, distal ends of the tool elements are pivotable towards each other. Therefore tissue, for example, can be grasped and held with grasping forceps by a pushing force being applied in the distal direction to the force transmitting member.

In particular, for the formation of scissors, it is advantageous for the end effector to comprise two tool elements and to be so constructed that as a result of a pulling force transmittable by the force transmitting member, distal ends of the tool elements are pivotable towards each other. In particular, such a configuration may also be extremely well-suited for the formation of grasping forceps.

To enable the at least one tool element to be mounted in a defined manner on the bearing shaft, it is advantageous for a proximal end of the at least one tool element to be in the form of at least one bearing jaw having a bearing shaft receptacle constructed so as to correspond to the bearing shaft.

The manufacture and construction of the end effector are particularly simple when the at least one bearing jaw is substantially parallelepipedal or cubical in shape. Preferably, the at least one bearing jaw is spaced from the force transmitting member. The spacing corresponds to at least an extent of the bearing jaw parallel to the longitudinal axis, but may also be two or three times larger.

Preferably, the at least one bearing jaw has an outer surface matching an inner contour of the shaft. In the case of an elongated sleeve-shaped or tubular shaft, the outer surface of the bearing jaw is preferably in the form of a spherical surface section. This enables pivoting of the tool element even when the outer surface of the at least one bearing jaw lies directly on an inner wall of the tubular shaft.

Preferably, a width of the at least one bearing jaw in a direction parallel to a pivot axis defined by the bearing shaft is approximately half as large as an inner diameter of the shaft. This allows two bearing jaws to be mounted next to each other on the bearing shaft, for example, in order to form an end effector with two tool elements which are pivotable relative to each other.

It is advantageous for an articulation member supporting section facing the at least one articulation member to be formed at a proximal end of the at least one tool element. The articulation member supporting section is preferably of flat configuration and defines a surface area. In particular, in the case of a so-called crossover arrangement of two flexure hinges and the associated tool elements, i.e., intersecting tool elements, it enables stabilization of the flexure hinges during gripping operations when high forces are transmitted through the flexure hinges. This case occurs particularly when the tool elements are subjected to a pulling force. In particular, in the case of intersecting tool elements, these can be subjected to pressure for opening, but to a pulling force for closing. The load resulting from a pulling force, in contrast to the load resulting from a pushing force or pressure, prevents a yielding, the so-called “buckling” of the resiliently flexible flexure hinges. However, the subjecting of the flexure hinges to a pulling force in the loaded state of the tool elements, i.e., particularly during gripping operations, may result in a so-called “tautening” of the flexure hinges. The negative consequence of this are high notch stresses in the initial and end areas of the flexure hinges, i.e., in particular, in the articulation members. Furthermore, the stiffness of the end effector in the loaded state is reduced by the “tautening”. The disadvantageous effect of the “tautening” increases as the opening angles of the tool elements increase. The articulation member supporting sections counteract these disadvantages, in particular, they help to reduce the “tautening” of the flexure hinges in the loaded state and support a natural bending line of the flexure hinges or articulation members. This can be achieved, in particular, by the articulation member supporting sections lying with surface-to-surface contact on the articulation members in a corresponding position of the tool elements. In particular, in gripping operations of intersecting tool elements with high forces and large opening angles, significant advantages are thereby achieved over articulation members that are not supported.

A particularly simple construction of the surgical instrument can be achieved by the at least one bearing jaw comprising the articulation member supporting section. In particular, the articulation member supporting section can then be integrated in the bearing jaw, and there is no necessity for any further elements or components on the instrument.

In order to make better use of the advantages of the articulation members which are at least flexible in a section thereof, it is expedient for the at least one articulation member, in a position of the tool element defining a normal position, to be undeformed and spaced from the articulation member supporting section. In particular, in the case of intersecting tool elements, these can then be opened by applying a pushing force or pressure. A supporting of the articulation members by the articulation member supporting section may occur, in particular, when an object or tissue is grasped with the tool elements and movement of these towards each other is thereby blocked. Only then is it optionally possible for the articulation member supporting section to come into contact with the at least one articulation member in order to support it in the described manner, which results in an advantageous reduction of notch stresses at the at least one articulation member and in a reduction of load on components in the loaded state of the tool elements, i.e., in particular, during gripping operations with large opening angles.

In accordance with a preferred embodiment, it may be provided that in a position of the tool element defining a pulled position, the articulation member supporting section is supported with surface-to-surface contact on the at least one articulation member, and, in the pulled position, the at least one tool element is swivelled out and blocked in the swivelled-out position. In particular, the articulation member supporting section can be so arranged and constructed that a supporting of the articulation member by the articulation member supporting section with surface-to-surface contact only occurs when the tool elements define or assume the pulled position. The supporting effect can therefore already occur, in dependence upon a respective loading situation of the tool elements, at opening angles of the tool elements below a prescribed target opening angle. In other words, the desired supporting effect described can occur at the flexure hinges, in particular, during gripping operations with large opening angles of the tool elements.

In the pulled position, the at least one tool element is expediently swivelled through at least 20° in relation to a normal position in which the at least one articulation member is undeformed. This configuration enables the described supporting effect to be achieved by means of the articulation member supporting section. Accordingly, below a swivel angle of 20° contact does not occur between the at least one articulation member and the articulation member supporting section. In the case of two tool elements which are pivotable relative to each other, a swiveling of 20° of a tool element in relation to the normal position corresponds in total to an opening angle between the tool elements of 40°.

In accordance with a further preferred embodiment, it may be provided that a first tool element comprises two first bearing jaws arranged in spaced relation to each other transversely to the longitudinal axis, and that a second tool element comprises a second bearing jaw mounted between the two first bearing jaws on the bearing shaft. A mirror-symmetrical arrangement of two tool elements is thereby achieved, in which, in particular, the second tool element can be mounted on the bearing shaft and thereby penetrate the first tool element or engage in a recess thereof. For this purpose, the first tool element optionally comprises an opening or a corresponding slot for the second tool element or a tool end thereof.

Advantageously, the at least one tool element has a tool end in the form of a cutter or in the form of a clamping jaw. End effectors can then be constructed in the form of cutting, clamping or grasping devices.

To construct a particularly small instrument, it is expedient for the tool end to have a material thickness ranging from 300 μm to 700 μm. In particular, the material thickness can define the thickness of a cutter.

Preferably, the tool end extends distally of a connecting area between the at least one articulation member and the at least one tool element. The at least one tool element can thereby be kept free of parts that obstruct a function of the instrument.

Body tissue can be easily cut, grasped or held, in particular, when the surgical instrument is constructed in the form of scissors, grasping forceps or tweezers.

In principle, it is conceivable for the surgical instrument to be made from any materials suitable for the manufacture of surgical instruments. However, the end effector is preferably made of a metal or a plastic material. In principle, manufacture from a plastic material, for example, polyetheretherketone (PEEK) allows manufacture of the end effector by a thermoforming process, for example, injection molding. The end effector manufactured from a metal, for example, an instrument steel, can be made particularly robust and therefore particularly small.

Preferably, an outer diameter of the shaft ranges from 1.5 mm to 8 mm.

It is expedient for it to range from 1.5 mm to 4 mm. Preferably, it is smaller than 3 mm. In particular, with the smallest aforementioned shaft outer diameters minimally invasive surgery can be carried out on the head or on small children without causing severe trauma.

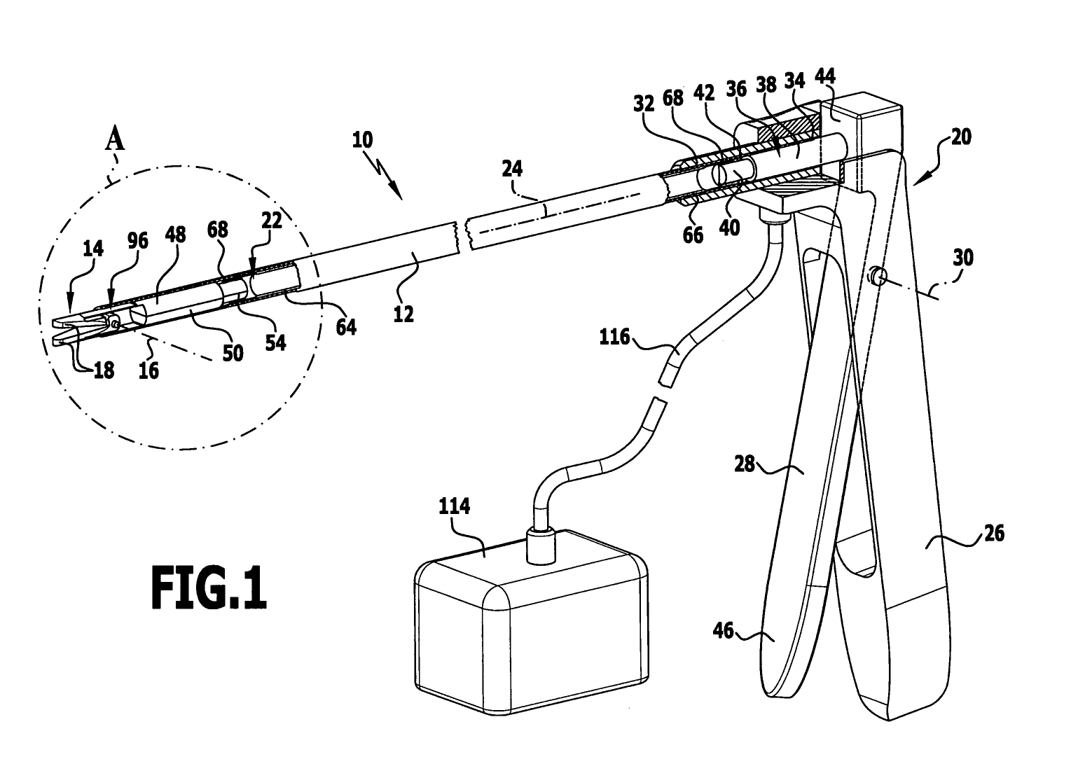

FIG. 1 shows a surgical instrument generally designated by reference numeral 10, comprising an elongated tubular shaft 12, an end effector 14 arranged at its distal end with two tool elements 18 in the form of clamping jaw-type jaw parts pivotable relative to each other about a pivot axis 16, and an actuating device 20 arranged at the proximal end of the instrument 10 for introducing and transmitting an actuating force onto a force transmitting member generally designated by reference numeral 22 for transmitting the actuating force onto the end effector 14 to move the tool elements 18.

The actuating device 20 comprises a grip part 26 projecting substantially transversely to a longitudinal axis 24 defined by the shaft 12. A grip element 28 is mounted on the grip part 26 for pivotal movement about a pivot axis 30 which is spaced from the longitudinal axis 24 but extends perpendicularly thereto. A proximal end of the shaft 12 is held in a guide sleeve 32 which projects in the distal direction from the grip part 26 and is arranged coaxially with the longitudinal axis 24. The shaft 12 may be fixedly or releasably connected to the guide sleeve 32.

A first piston 36 is mounted in the proximal end area 34 of the shaft 12 for displacement parallel to the longitudinal axis 24. It comprises a first piston section 38 whose outer diameter matches an inner diameter of the shaft 12. A second piston section 40 facing the distal end projects coaxially with the longitudinal axis 24 from the first piston section 38. The outer diameter of the second piston section 40 is somewhat smaller than the inner diameter of the shaft 12, thereby forming a ring space 42 surrounding the second piston section 40. A pressure surface 44 formed at the level of the longitudinal axis 24 and facing in the distal direction from the grip element 28 lies against an end face, facing in the proximal direction, of the piston 36 and can move the piston 36 in the distal direction as a result of pivotal movement of the grip element 28 about the pivot axis 30. In particular, an articulated connection of the actuating device 20 and the piston 36 of the force transmitting member 22 is thereby also effected. Optionally, a resetting element, not shown, may be provided on the actuating device 20. The resetting element transfers the grip element 28 to a normal position when no pressing force is being applied to it. In the normal position, the pressure surface 44 preferably assumes its most proximal position, which, at the same time, means that an actuating area 46, projecting furthest from the longitudinal axis 24, of the grip element 28 projects maximally far in the distal direction and faces in this direction.

The force transmitting member 22 is of multipart construction and comprises, inter alia, the piston 36. The force transmitting member 22 also comprises a further piston 48 which defines a distal end of the force transmitting member 22. The piston 48 which forms a force transmitting section 49, which is connected to the force transmitting member 22 and forms a distal end thereof, is of substantially identical construction to the piston 36 and comprises a first cylindrical piston section 50 having an outer diameter which corresponds to an inner diameter of the shaft 12, so that the piston 48 is mounted for movement in the shaft parallel to the longitudinal axis 24 substantially free of play. From an end face 52, facing in the proximal direction, of the first piston section 50 there projects coaxially with the longitudinal axis 24 a second piston section 54 facing in the proximal direction. The length of the second piston section 54 is about one third of the length of the first piston section 50. A ring space 56 is formed so as to surround the second piston section 54. An end face 58, facing in the proximal direction, of the second piston section 54 defines a pressure surface. Edges of the cylindrical second piston section 54 are rounded off.

A fluid section generally designated by reference numeral 60 extends between the two pistons 36 and 48. It comprises a tube 64 substantially lining an inner wall 62 of the shaft between the second piston sections 40 and 54. The free proximal and distal ends 66 of the tube 64 are each closed with a sealing element 68 of identical construction. Therefore, the following description of the coupling of the pistons 36 and 48 to the fluid section 60 will be limited to the coupling of the piston 48 to the fluid section 60, which define a so-called piston-sealing element assembly.

The tube 64 is preferably fixedly connected to the shaft 12 and non-rotatable relative thereto about the longitudinal axis 24. It itself can form a shaft if the shaft 12 is dispensed with. The sealing element 68 is formed directly on the tube 64 or is fixedly connected thereto and directly follows the respective end 66. The sealing element 68 comprises a first, substantially sleeve-shaped section 70 which is barely twice as long as a length of the second piston section 54 parallel to the longitudinal axis 24. A free end, facing away from the end 66, of the section 70 is closed by a second section 74 of the sealing element 68. The section 74 extends substantially transversely to the longitudinal axis 24 and defines a surface area corresponding substantially to the size of an inside free cross-sectional area of the shaft 12. The sealing element 68 is flexible, but inelastic and constructed in the form of a fluid-tight membrane forming a so-called rolling bellows. The sealing element 68 has a ring-shaped edge 76 which faces away from the respective piston and, in the given case, is fixedly connected to the end 66 of the tube 64 and therefore indirectly fixedly connected to the inner wall 62.

The tube 64 with the sealing elements 68 formed on both sides defines a fluid space 78 which is filled with a fluid 80, preferably an incompressible fluid. The fluid space 78 is preferably filled in a gas-free manner, which means that, in particular, no air bubbles or gas bubbles are contained in the fluid space 78. In any case, the fluid space 78 is permanently filled with the fluid 80 and is preferably leak-free. A volume of the fluid 80 corresponds approximately to a volume defined by the tube 64, i.e., the volume defined by the product of the inside cross-sectional area of the tube 64 and its length.

The second sections 74 of the sealing elements 68 may be optionally connected to the second piston sections 40 and 54, for example, to the end face 58 of the second piston 54. The sealing element 68 has a wall thickness which is less than 0.5 mm, preferably less than 0.1 mm, and, depending on the choice of material, may also be less than 0.05 mm. The ratio of a length of the first section 70 to a diameter of the second section 74, in the embodiment shown in the Figures, is approximately 2. It may, however, be smaller than 1, in particular, also smaller than 0.5 or 0.2.

The sealing element 68 is constructed such that the second section 74 can be turned inside out, thereby forming a double layered rolled sealing section 82 which faces away from the edge 76 and is closed in a direction facing away from the edge 76. This means that the first section 70 is folded back onto itself or turned over in the direction on the longitudinal axis 24, so that a first part of the first section 70 lies against the shaft 12, and a second part of the first section 70 against an outer surface of the second piston section 54. The sealing section 82 as a whole enters the ring space 56 without becoming jammed in it. This makes it possible for the second section 74 to be movable parallel to itself in the direction of the longitudinal axis 24, in both the distal and the proximal directions. Upon movement of the second section 74, for example, in the distal direction, the partially turned-over first section 70 rolls off and can be completely rolled off in a most distal position of the piston 48 and line the inner wall 62 in the form of a cylindrical section of the sealing element 68. Conversely, the second section 74 can be moved up to the edge 76, with the first section 70 then being turned inside out so that the sealing section 82 has approximately half the length of the first section 70. In principle, it is conceivable to so select the dimensions of the parts relative to one another that the second section 74 can be pushed in the proximal direction over the edge 76 in the direction towards the actuating device 20. This is, in principle, possible until the first section 70 is rolled off completely again. This results in theory in a maximum path of displacement of the piston 48, which corresponds to twice the length of the first section 70.

The described assembly comprising piston 48 and sealing element 68 is also provided in like manner at the piston 36. This is indicated schematically in FIG. 1. The pistons 36 and 48 are preferably arranged such that in a normal position of the fluid section 60, the second piston sections 40 and 54 rest against the second sections 74, which are both respectively arranged approximately level with the edges 76. As described above, the fluid space 78 is then filled in a bubble-free manner with the fluid 80, which, in particular, also fills out the ring space-like sections in the area of the sealing sections 82.

When the actuating area 46 of the grip element 28 is pivoted in the proximal direction, the pressure surface 44 is moved in the distal direction and also pushes the piston 36 forwards in the distal direction. At the same time, the second section 74, lying against the second piston section 40, of the sealing element 68 is moved in the distal direction, more specifically, distally of the edge 76. Owing to the incompressibility of the fluid 80, the piston 48 is moved in the distal direction as a result of movement of the piston 36. The sealing section 82 thereby rolls off, the part of the sealing section 82 lying against the inner wall 62 of the shaft 12 and supported thereon becomes longer and longer, and the part of the sealing section 82 lying against the second piston section 54 and supported thereon becomes shorter and shorter until the first section 70 has rolled off completely and the second section 74 forms a distal end of the sealing element 68. In a converse manner, a movement of the piston 48 in the proximal direction results in a movement of the piston 36 in the proximal direction, too. If the sections 74 are connected to the respective second piston sections 40 and 54, the piston 48 can also be pulled in the proximal direction by a movement of the piston 36 in the proximal direction.

In the instrument 10, the end effector 14 is formed directly on the piston 48. In principle, it is possible to construct the piston 48 and the end effector 14 in one piece with the tool elements 18. However, the end effector 14 is easier to manufacture in connection with the piston 48 when two identical parts are formed, which are held on the shaft by a pin defining the pivot axis 16 and a bearing shaft 84. The pin is mounted, for example, in bores oriented coaxially with the pivot axis 16 in a wall of the shaft 12. There are mounted on the bearing shaft 84 proximal end sections 86 of the tool elements, which form substantially cubical bearing jaws 87 and are provided with a bore 88 formed and arranged so as to correspond to the bearing shaft 84. The two end sections 86 of the tool elements 18 are arranged next to each other on the bearing shaft 84. Outer surfaces 89 of the bearing jaws 87 that face away from each other are convexly curved and preferably define spherical surfaces with a radius corresponding to a radius of the shaft 12.

A tool end 90 of the respective tool element 18 is rigidly connected to the respective end section 86. The bearing jaws 87 are approximately only half as wide as a proximal end of the tool end 90 so that if the tool ends 90 overlap, the bearing jaws 87 are mounted next to each other for pivotal movement on the bearing shaft 84.

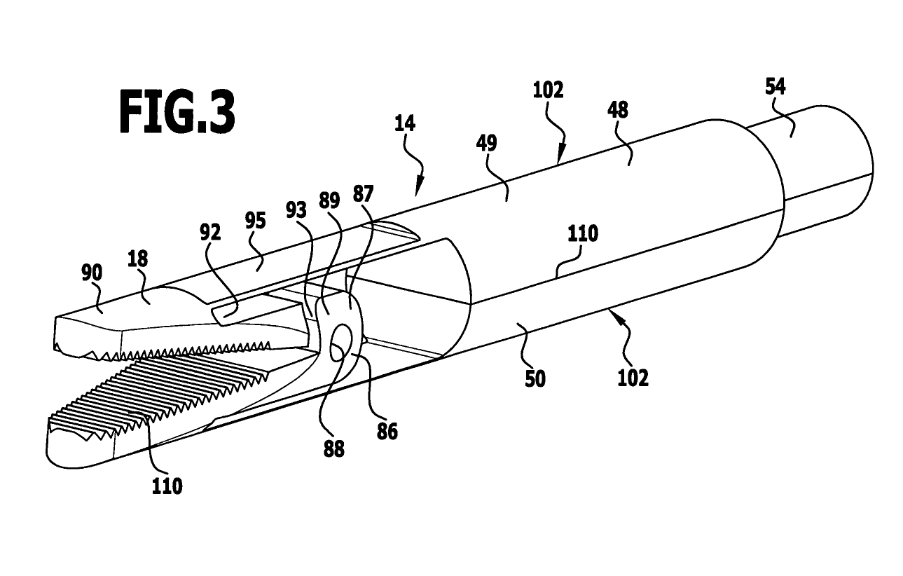

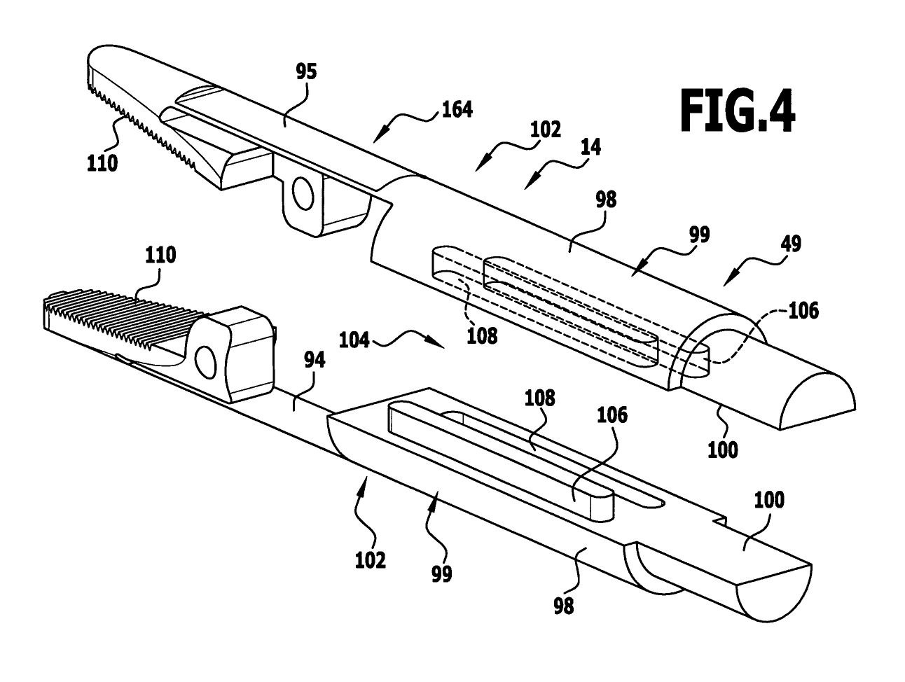

In the present instrument 10, tool ends 90 are in the form of clamping jaws 91 which are approximately as wide as a diameter of the shaft 12 and whose inner surfaces that face each other, as shown in FIGS. 3 and 4, may be optionally provided with a transverse toothing 110 which may have a toothing height ranging from 50 μm to 400 μm, preferably 200 μm to 300 μm. Approximately halfway between the distal end of the tool end 90 and the pivot axis 16 there is formed by a gap 92 a flat, elongated web 94, which defines an outer surface of the tool element 18 and whose proximal end is formed in one piece with the piston 48. The web 94 is flexible and elastically bendable and forms an articulation member 95 in the form of a flexure hinge 96. The web 94 extends approximately the same distance in the distal as in the proximal direction in relation to the pivot axis 16. An opening is thereby defined, which is distally delimited by the bearing jaws 87, proximally by the first piston section 50 and radially by the opposed articulation members 95. A spacing between a proximal end of the bearing jaws 87 and a distal end of the first piston section 50 is approximately two or three times greater than an extent of the bearing jaws 87 in a direction parallel to the longitudinal axis 24. To achieve optimum movability of the tool elements 18 and prevent jamming thereof on the bearing shaft 84, proximal ends of the tool ends 90 are separated by a narrow gap from the bearing jaw 87 of the respective other tool element 18.

The manufacture of both the piston 48 and the end effector 14 is simplified by two identical end effector parts 102 being formed, which can be fitted together by rotation about the longitudinal axis 24. To this end, the piston 48 is assembled from two piston halves 98, each forming a section half 99 of the force transmitting section 49. The section halves 99 define connecting surfaces 100 containing the longitudinal axis 24, extending parallel to the webs 94 and dividing the piston 48 lengthwise in halves with its first and second piston sections 50 and 54. A connecting device generally designated by reference numeral 104, respectively carrying two connecting elements 106 and 108, serves to connect the two end effectors parts 102. The connecting element 106 is in the form of an elongated projection extending parallel to the longitudinal axis 24. The other connecting element 108 is in the form of a recess corresponding to the projection for engagement of the connecting element 106 therein with a positive connection. The connecting elements 106 and 108 are respectively arranged on the connecting surfaces 100 such that in a connected position the two section halves 99 form the force transmitting section 49. Optionally, the piston halves 98 are fixedly connected to each other, for example, by adhesive bonding or welding.

The articulation member 95 has a material thickness which, in dependence upon the diameter of the force transmitting section 49 ranges from approximately 50 μm to 700 μm. In the case of an inner diameter of the shaft 12 of approximately 2 mm, the thickness of the web 94 is approximately 100 μm.

The mode of operation of the end effector 14 will be described in detail hereinbelow.

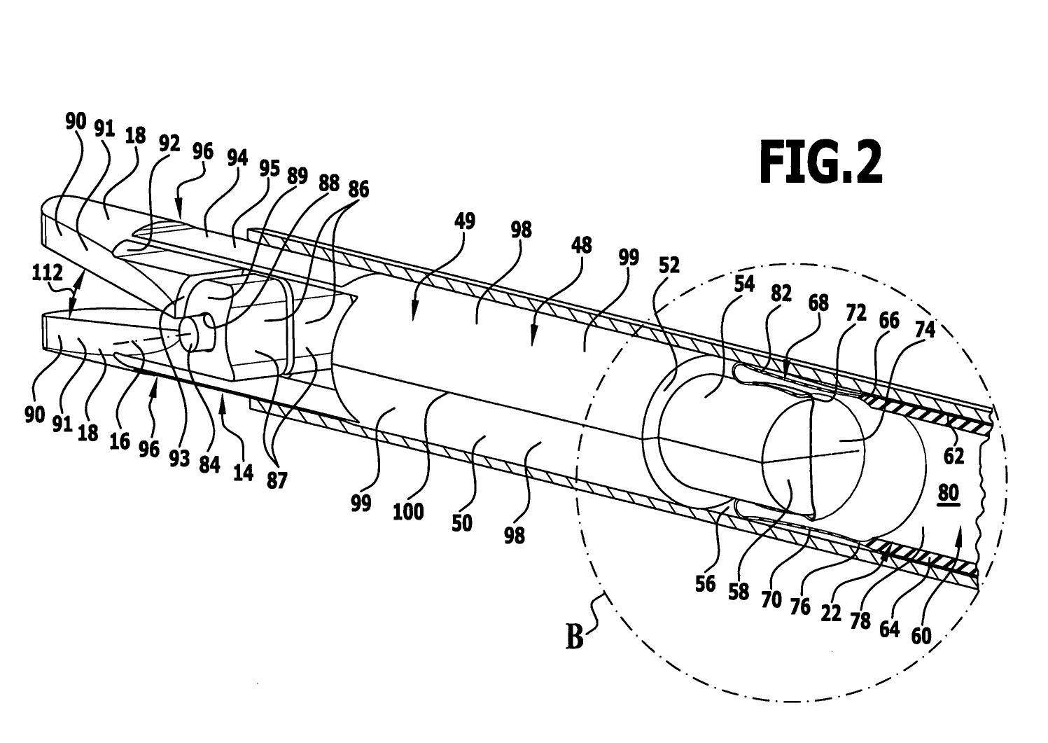

FIGS. 1 and 2 show the end effector 14 in a normal position. The tool ends 90 are spread open somewhat and define between them an opening angle 112 of approximately 30°. In the normal position, the webs 94 extend parallel to the longitudinal axis 24. If the piston 48 is moved in the proximal direction, the webs 94 are also pulled in the proximal direction. Since the tool ends 90 are rigidly mounted for pivotal movement about the bearing shaft 84, the web 94 and therefore the flexure hinge 96 undergo deformation, so that the tool ends 90 are pivoted outwards away from the longitudinal axis 24. The opening angle 112 can thereby be increased, which facilitates the grasping of tissue with the end effector 14. Appropriate manufacture also enables deformation of the web 94 to some extent in a normal position, so that the respective tool ends 90 are pivoted further apart without forces being introduced through the piston 48.

When the piston 48 is moved in the distal direction, the web 94 is also displaced in the distal direction. Since the tool ends 90 have no other movement possibility than to be pivoted towards each other, this occurs with corresponding deformation, i.e., elastic bending of the web 94 and therefore of the flexure hinge 96. A displacement path of the piston 48 is delimited by the tool ends 90 whose inner surfaces, in a closed position, not shown in the Figures, lie against each other or lie substantially against each other.

The flexure hinges 96 enable an almost play-free mounting and force transmission from the force transmitting member 22 onto the tool elements 18 in order to move these, i.e., in the present case, in order to pivot these about the pivot axis 16.

As described above, the piston 36 may be provided for moving the piston 48 in the distal and proximal directions. Optionally, the piston 36 with the associated sealing element 68 can also be dispensed with if an alternative fluid feed device 114 is provided, for example, in the form of an external fluid pump which is connected in a fluid-tight manner to a proximal end of the shaft 12 and the tube 64 arranged therein through a feed line 116. With the fluid feed device 114 the fluid 80 can pressed into the proximal end of the shaft 12 and the piston 48 thereby moved in the distal direction. Owing to the elastically resilient flexure hinges 96, a removal or reduction of the pressure in the fluid 80 then results in a swiveling of the tool elements 18 back into their normal position and, therefore, due to the connection of the piston 48 to the tool elements 18 by means of the webs 94, in a return movement of the piston 48 in the proximal direction.

An actuation of the fluid feed device 114 can be brought about by, for example, the pivotably mounted grip element 28. Preferably, an electric, electronic or electromechanical circuit element is provided for actuating the fluid feed device 114 as a result of a pivotal movement of the grip element 28, for example, in the form of a switch, pushbutton or electronic switching element, not shown, such as an optical sensor or an inductive or capacitive proximity sensor, which is arranged on the actuating device 20 for detecting a movement of the grip element 28 relative to the grip part 26.

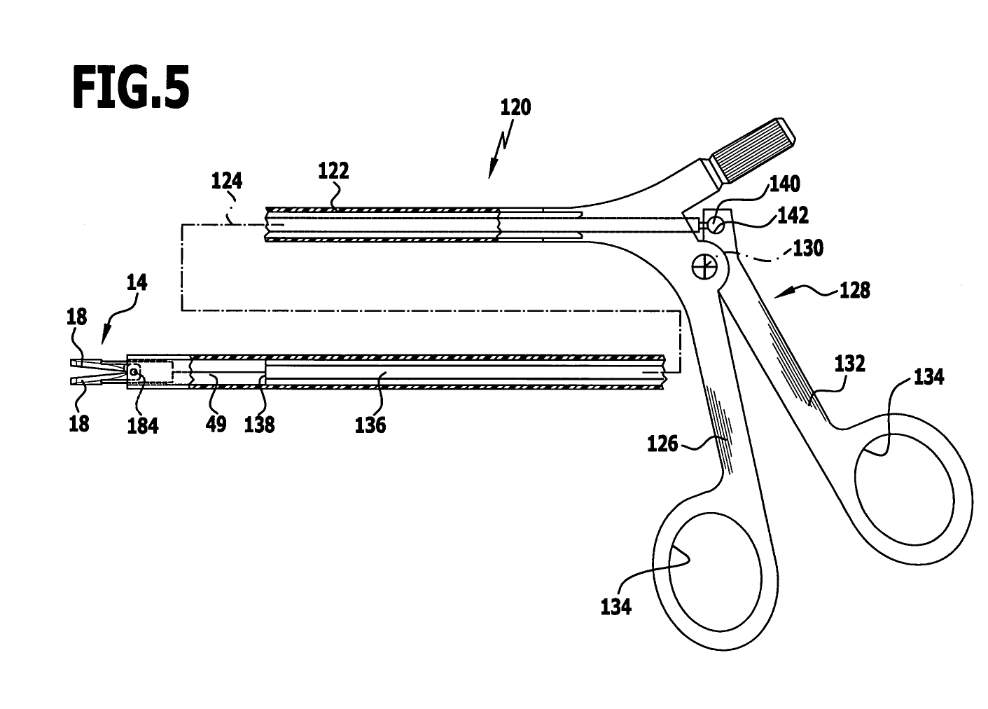

FIG. 5 shows an alternative embodiment of a surgical micro tubular shaft instrument. It comprises an elongated tubular shaft 122, at the distal end of which the end effector 14, which is known from the instrument 10 and is described hereinabove, is mounted. For details of the construction of the end effector 14 reference is therefore to be had to the above description.

A proximal end of the shaft 122 is bent approximately at right angles to a longitudinal axis 124 of the shaft 122 and forms a first grip part 126 of an actuating device 128. A second grip part 132 is mounted for pivotal movement relative to the grip part 126 about a pivot axis 130 which is oriented transversely to but spaced from the longitudinal axis 124. Free ends of the grip parts 126 and 132 are each provided with a finger ring 134 for insertion of one or more fingers of an operator.

Mounted for pivotal movement in the shaft parallel to the longitudinal axis 124 is a force transmitting member in the form of a push-and-pull rod 136 whose distal end 138 is fixedly connected to the force transmitting section 49 of the end effector 14. A proximal end of the push-and-pull rod 136 is in the form of a spherical head 140, which is mounted in a corresponding receptacle 142 of a section of the grip part 132 that projects over the pivot axis 130 in order to form a ball-and-socket joint-type connection of the push-and-pull rod 136 with the grip part 132. By pivoting the grip part 132 relative to the grip part 126 the push-and-pull rod 136 can be moved in the distal direction in order to close the tool elements 18 and in the proximal direction in order to open the tool elements 18.

The instrument 120 shown in FIG. 5 serves as an example of how it is possible to combine the end effector 14 described in conjunction with the instrument 10, and also all other end effectors described hereinbelow, with so-called conventional tubular shaft instruments, i.e. with instruments having a bar-shaped push-and-pull rod 136 or the like as force transmitting member. The bearing shaft 84 is held in corresponding blind holes or bores of the shaft 122.

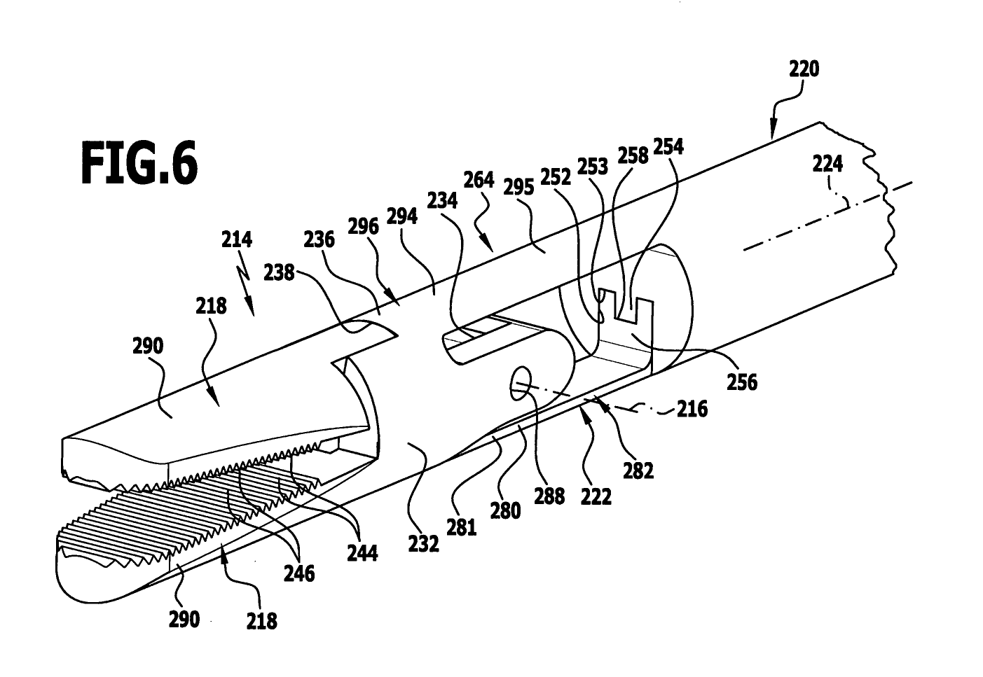

A further embodiment of an end effector which can be provided on both the instrument 10 and the instrument 120 is shown by way of example in FIGS. 6 to 9 and generally designated by reference numeral 214.

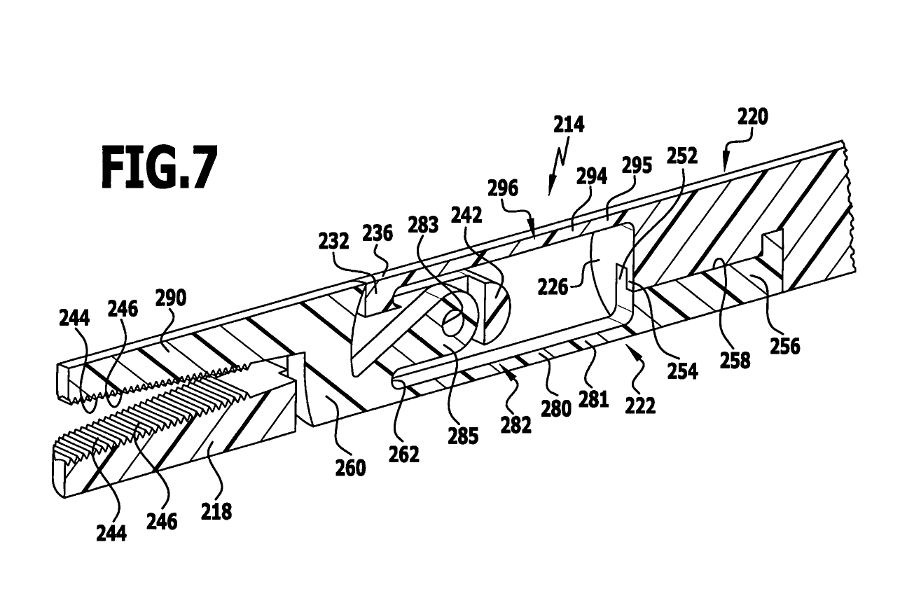

The end effector 214 comprises two tool elements 218 mounted for pivotal movement relative to each other about a pivot axis 216, which is defined by a bearing shaft, not shown, which is held on a shaft. The end effector 214 is overall of two-part construction and comprises a first end effector part 220 and a second end effector part 222. Unlike the end effector parts 102, the two end effector parts 220 and 222 are not of identical construction. Rather, each end effector part viewed by itself, also unlike the end effector parts 102, is of symmetrical construction in relation to a mirror plane extending perpendicularly to the pivot axis 216 and containing the longitudinal axis 224 of the end effector 214. A sectional view of the end effector 214 taken along this mirror plane is shown in FIG. 7.

The end effector part 220 comprises a cylindrical force transmitting section 249 which may also be referred to as piston. A proximal end thereof is shaped in analogy with the piston 48 for use in an instrument 10 or is connected to a push-and-pull rod 136 as in instrument 120. The force transmitting section 249 defines an end face 226 facing in the distal direction. From the force transmitting section 249 there projects in the distal direction a web 294 which defines a flexible articulation member 295 for formation of a flexure hinge 296. An outer surface 228, facing away from the longitudinal axis 224, of the web 294 forms a continuation of a cylindrical outer surface of the force transmitting section 249. An inner surface 230, facing towards the longitudinal axis 224, of the web 294 is flat.

The articulation member 295 is formed on a cylindrical pull section 232 of the tool element 218. A gap extending parallel to the web 294 separates it from two bearing jaws which, in turn, are spaced from each other by a gap 234. The bearing jaws 287 face in the proximal direction and are provided coaxially with the pivot axis 216 with a bore 283 through which a pin-shaped bearing shaft can be inserted in order to mount the tool element 218 of the end effector part 220 for pivotal movement on a shaft. Somewhat distally of a connecting area between the web 294 and the pull section 232, the pull section 232 is provided with a through-opening 238 which extends through the pull section 232 in approximately parallelepipedal shape symmetrically with the plane of symmetry and transversely to the longitudinal axis 224 and opens up an end face 240 of the pull section 232 which faces in the distal direction. The gap 234 and the through-opening 238 are connected to each other. The gap 234 does not separate the two bearing jaws 287 completely, but ends a short distance before a proximal end thereof, so that a connecting web 242 connecting the bearing jaws 287 and facing in the proximal direction is formed. A tool end 290 facing in the distal direction projects from the pull section 232, more specifically, such that a virtual extension thereof in the proximal direction would be diametrically opposed to the web 294. A clamping surface 244 facing towards the longitudinal axis 224 is provided with a transverse toothing 246 which may have a height in the range of between 20 μm and 100 μm.

Serving to connect the end effector part 220 to the end effector part 222 is a connecting device 248 with a first connecting element 252, which is formed on the force transmitting section 249, more specifically in the form of a parallelepipedal recess which is open in a radial direction facing away from the longitudinal axis 224. From a bottom 253 of the recess there projects an elongated parallelepipedal strip 254 which in a direction parallel to the longitudinal axis 224 is somewhat shorter than the recess, but ends flush with the end face 226. A width transverse to the longitudinal axis 224 of the connecting element 252 corresponds to a width of the through-opening 238 and of the gap 234.

The connecting device 248 comprises a second connecting element 256 which is of such shape that it fills out the recess defined by the connecting element 252 completely. It is therefore overall of approximately parallelepipedal shape and has an outer surface section which complements the force transmitting section 249 to form a cylinder. To accommodate the strip 254 there is formed on the connecting element 256 a groove 258 which is closed proximally. The strip 254 can therefore engage the groove 258, but a movement of the two connecting elements 252 and 256 relative to each other parallel to the longitudinal axis 224 is prevented.

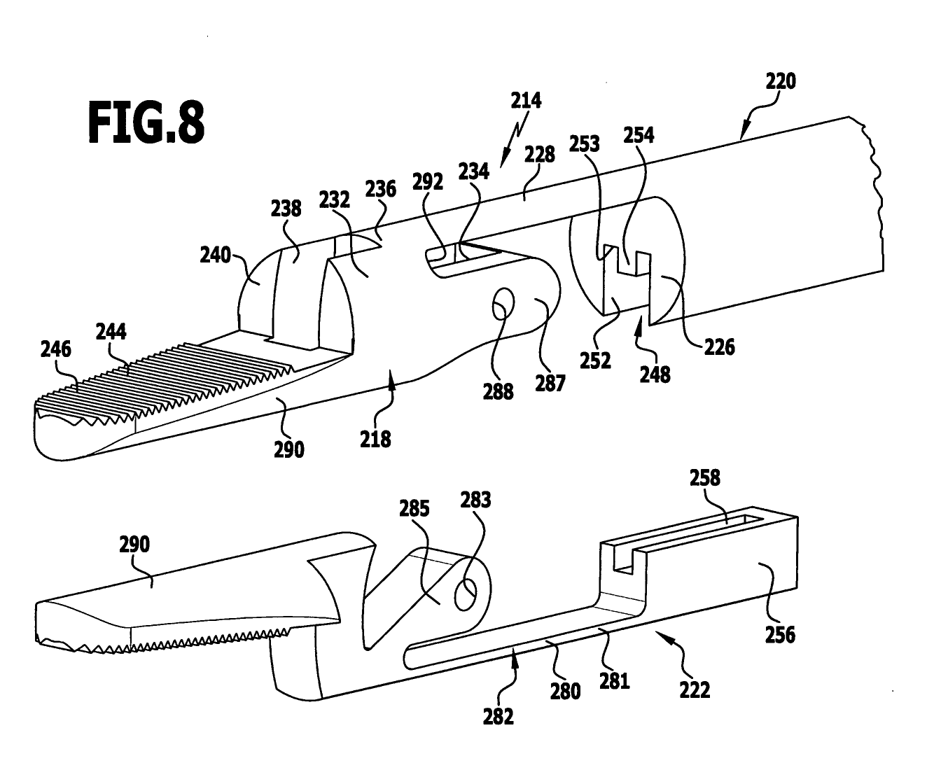

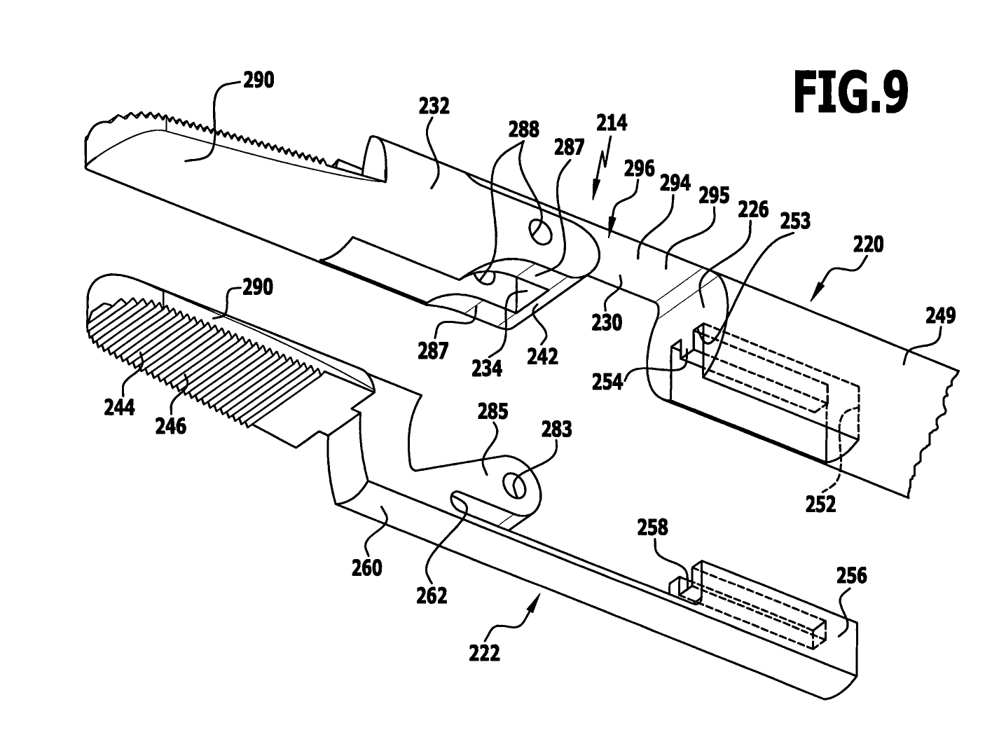

Diametrically opposed to the web 294, there extends in the distal direction from the connecting element 256 a web 280 defining an articulation member 281, which forms a flexure hinge 282 for transmitting push-and-pull forces from the force transmitting section 249 onto the tool element 218 comprised by the second end effector part 222. The web 280 ends at a jaw-shaped pull section 260, from which there projects in the proximal direction a bearing jaw 285, obliquely facing the longitudinal axis 224, which has a bore 283. The bearing jaw 285 is separated from the web 280 by a short gap 262 extending in the distal direction somewhat beyond the pivot axis 216. The pull section 260, the web 280 and the bearing jaw 285 are all of identical width, and their width corresponds to a width of the connecting element 256 and therefore to a width of the through-opening 238. This makes it possible, for assembly of the end effector 214, to assemble the separately manufactured end effector parts 220 and 222, as shown in FIGS. 8 and 9, in such a way that by inserting the connecting element 256 into the through-opening 238 from above, i.e., facing the clamping surface 244 and the longitudinal axis 224, the connecting element 256 can be pushed through the through-opening 238 until it projects on the other side. The end effector parts 220 and 222 are then pivoted relative to each other in such a way that the connecting element 256 can engage the connecting element 252. The bores 283 and 288 are then in alignment, and a tool end 290 extending in the distal direction from the pull section 260, which then assumes a normal position as shown in FIGS. 6 and 7, is arranged symmetrically and opposite the other tool end 290. The end effector 214 can then be fixed with a bearing shaft, not shown, on a shaft of the instrument. Optionally, the end effector parts 220 and 222 can be adhesively bonded and/or welded in the area of the connecting device 248. If the force transmitting section 249 is guided in a sleeve-shaped shaft, this prevents separation of the two parts, so that adhesive bonding or welding is not absolutely necessary.

Owing to the penetrating or engaging arrangement of the tool elements 218, the end effector 214 is constructed such that a pulling force introduced by a force transmitting member results in a closing movement of the end effector, i.e., the clamping surfaces 244 are pivoted against each other. A movement of the force transmitting section 249 in the distal direction results in a pivoting of the tool elements 218 away from the longitudinal axis 224, so that an opening angle between the clamping surfaces 244 of the tool elements 218 is increased again. The described closing movement of the tool elements 218 as a result of a pulling force is enabled, in particular, because the points of application of the articulation members 281 and 295 respectively lie, in relation to the pivot axis 216, on other sides than the connecting areas of the tool ends 290 on the pull sections 232 and 260, respectively.

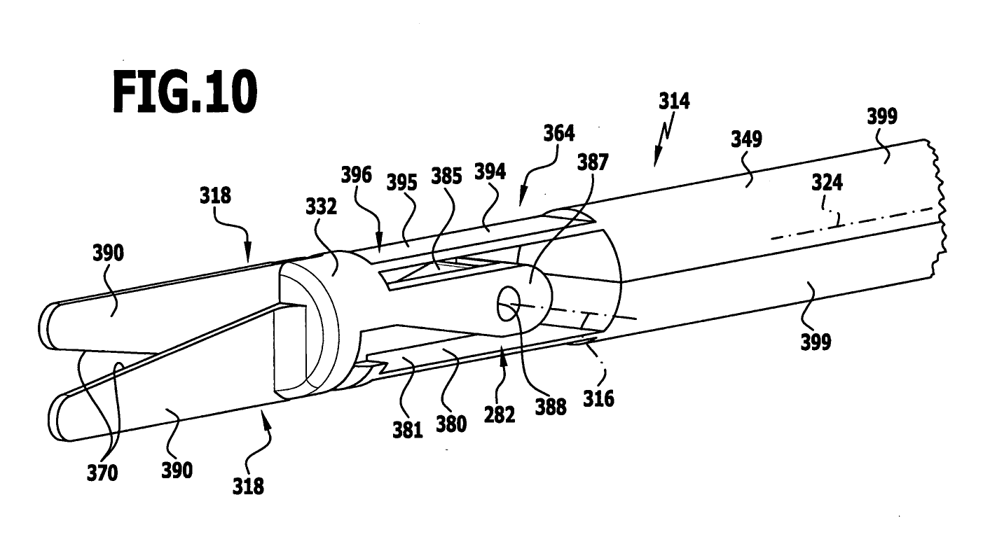

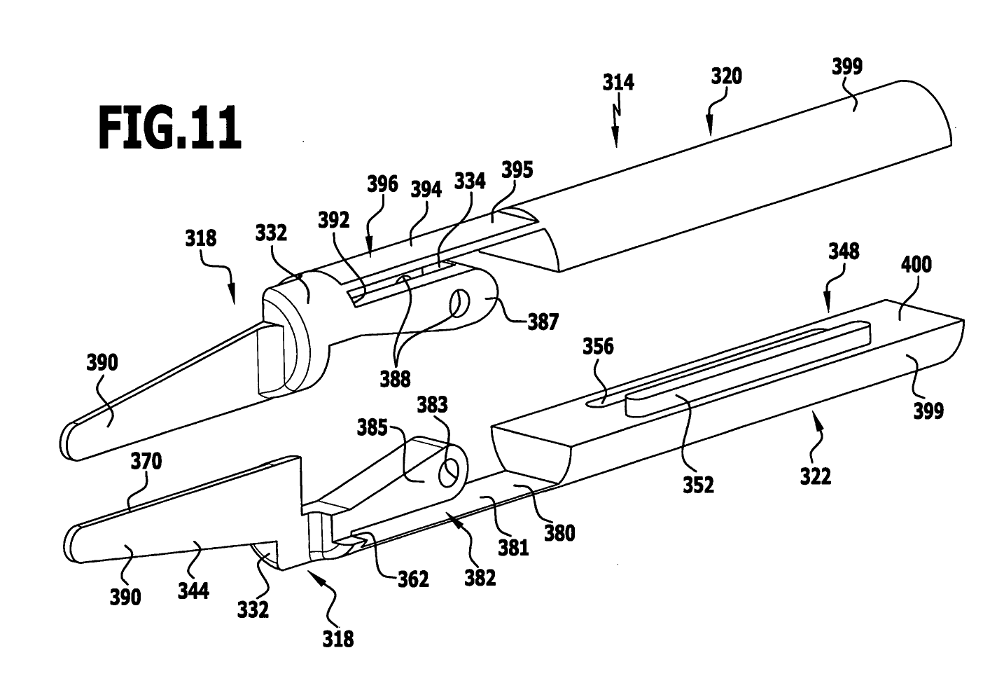

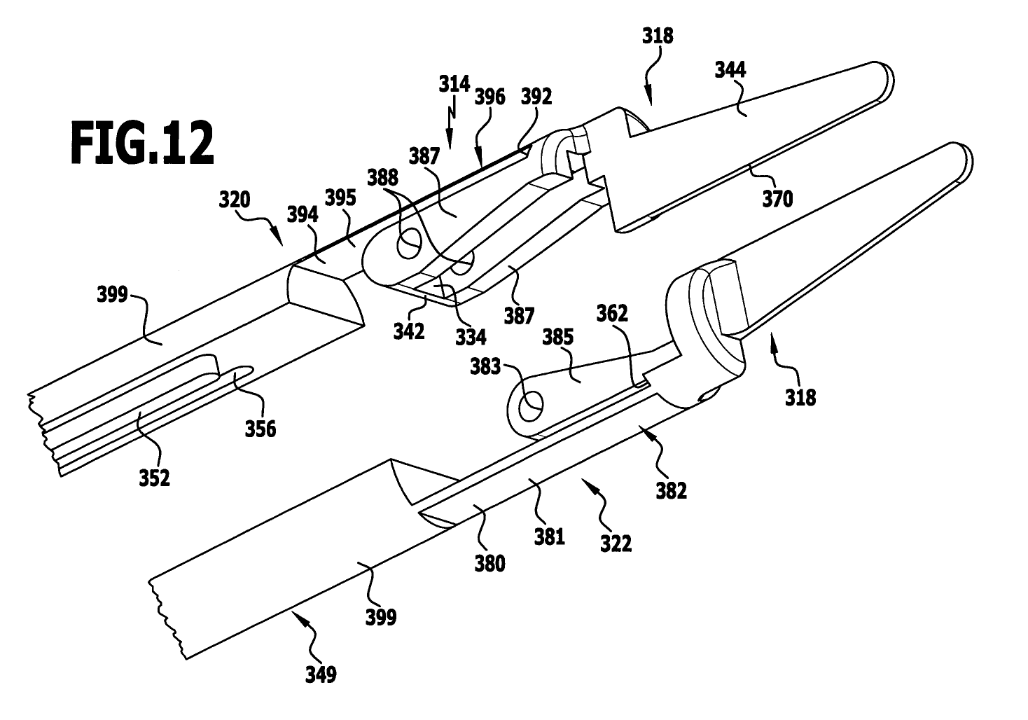

A further embodiment of an end effector constructed in the form of a cutting device is shown by way of example in FIGS. 10 to 12 and generally designated by reference numeral 314. The construction of the end effector 314 is essentially brought about by combining the configurations of the end effectors 14 and 214. The end effector 314 is also of two-part construction and comprises two end effector parts 320 and 322 which are not of identical construction. Distal ends thereof are formed by tool ends 390 in the form of cutters sliding on each other, which may have a material thickness ranging from 200 μm to 800 μm.

The force transmitting section 349 is of overall cylindrical construction and comprises two section halves 399 of identical construction. Like the section halves 99 of the end effector 14, these are connected to each other by a connecting device 348. This comprises on each connecting surface 400 of the section halves 399, which in the assembled state lie against each other, a first connecting element 352 in the form of a projection extending parallel to the longitudinal axis 324 and projecting towards the connecting surface of the respective other section half 399. There is formed in each section half 399 parallel to the connecting element 352 a further connecting element 356, more specifically, in the form of a recess which corresponds to the projection. When the section halves 399 are connected to each other, a connecting element 352 respectively engages in a positively locking manner the other connecting element 356 of the respective other section half 399, thereby forming overall the cylindrical force transmitting section 349.

From each section half 399 there respectively extends a narrow web facing in the distal direction, more specifically, a web 394 from the end effector part 320 and a web 380 from the end effector part 322. The webs 394 and 380 respectively form strip-shaped articulation members 395 and 381, respectively, for the formation of flexure hinges 396 and 382, respectively. The webs 394 and 380 respectively continue into a pull section 332. The pull sections 332 of both end effector parts 320 and 322 are of identical construction. The approximately half-cylindrical pull sections 332 with the tool ends 390 formed thereon can be fitted together by rotation about the longitudinal axis 324 through 180°. The tool ends 390 are arranged on the pull sections 332 so as to extend approximately parallel to each other and define cutting edges 370 sliding on each other in the manner that is customary for cutting devices.

For pivotal mounting of the tool elements 318 on a bearing shaft, not shown, which is mountable on a shaft and defines a pivot axis 316 transverse to the longitudinal axis 324, there are formed by a gap 392 extending parallel to the web 394 two bearing jaws 387, which are spaced in the direction of the pivot axis 316 from each other by a gap 334. Proximally, the bearing jaws 387 like the bearing jaws 287 are connected to each other by a connecting web 342.

There is arranged on the pull section 332 of the end effector part 322 a single bearing jaw 385 which projects in the proximal direction and is separated by a gap 362 extending parallel to the web 380. A width of the bearing jaw 385 corresponds to a width of the gap 334 so that the bearing jaw 385 can engage between the bearing jaws 387. The bearing jaws 385 and 387 are each provided with bores 383 and 388, respectively, which are aligned coaxially with the pivot axis 316.

Except for the construction of the respective bearing jaws 385 and 387, respectively, the end effector parts 320 and 322 are of identical construction and can be fitted together by rotation about the longitudinal axis 324 through 180°. The arrangement of the bearing jaws 385 and 387, on the other hand, is mirror-symmetrical in relation to a mirror plane perpendicularly intersecting the pivot axis 316 and containing the longitudinal axis 324. The pull sections 332 are each formed by a short half-cylindrical section, with cut lines of cut surfaces of the pull sections that lie against each other continuing into inner surfaces 344 of the tool ends 390 whose free edges form the cutting edges 370.

For cutting with the end effector 314, the force transmitting section 349 must be moved in the proximal direction. Accordingly, a cutting procedure can be brought about by transmitting a pulling force.

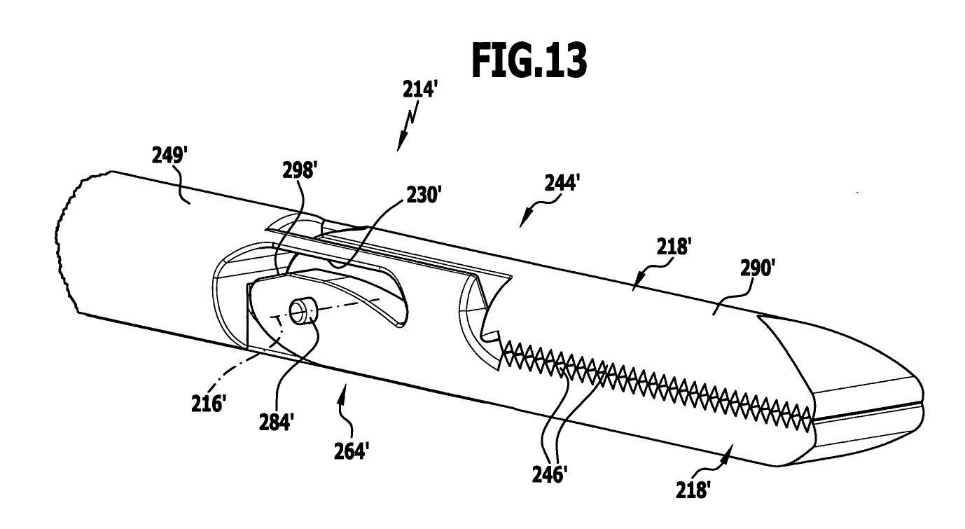

A further embodiment of an end effector, which may be provided on both instrument 10 and instrument 120, is shown by way of example in FIGS. 13 to 16 and generally designated by reference numeral 214′. The end effector 214′ corresponds in its basic structure to the end effector 214 described in conjunction with FIGS. 6 to 9. For reasons of clarity, identical parts are therefore given the same reference numeral with the addition of a ′.

The end effector 214′ comprises two tool elements 218′ mounted for pivotal movement relative to each other about a pivot axis 216′ defined by a bearing shaft 284′ held on a shaft. The end effector 214′ is overall of two-part construction and comprises a first end effector part 220′ and a second end effector part 222′. Unlike the end effector parts 102, the two end effector parts 220′ and 222′ are not of identical construction. Rather, the end effector part viewed by itself, also unlike the end effector parts 102, is symmetrical in relation to a mirror plane extending perpendicularly to the pivot axis 216′ and containing the longitudinal axis 224′ of the end effector 214′.

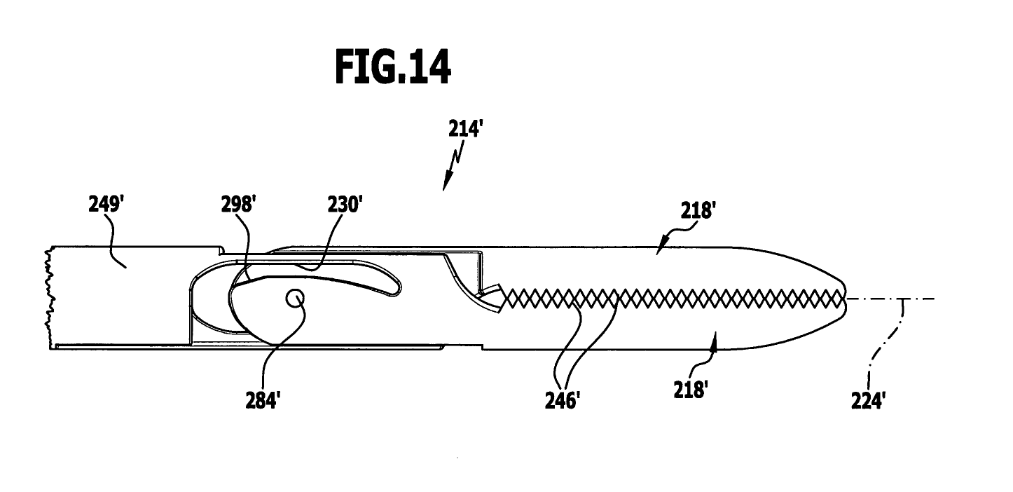

The end effector part 220′ comprises a cylindrical force transmitting section 249′, which may also be referred to as or may be a piston. A proximal end thereof is formed in analogy with the piston 48 for use in an instrument 10 or is connectable to a push-and-pull rod 136 as in the instrument 120. The force transmitting section 249′ defines an end face 226′ facing in the distal direction. From the force transmitting section 249′ there project in the distal direction two webs 294′ extending parallel to each other and to the longitudinal axis 224′ and spaced from each other. The webs 294′ define flexible articulation members 295′ for formation of a flexure hinge 296′. The articulation members 295′ also extend parallel to the longitudinal axis 224′ when the tool elements 218′ assume a normal position shown, for example, in FIGS. 13 and 14, in which the articulation members 295′ are not deformed and the clamping surfaces 244′ of the tool elements 218′ lie against each other. An outer surface 228′, facing away from the longitudinal axis 224′, of the webs 294′ forms a continuation of a cylindrical outer surface of the force transmitting section 249′. An inner surface 230′, facing the longitudinal axis 224′, of the respective web 294′ is flat at least in a section thereof.

The articulation member 295′ is formed on a cylindrical pull section 232′ of the tool element 218′. A gap 292′ extending parallel to the webs 294′ separates these from two bearing jaws 287′ which, in turn, are spaced from each other by a gap 234′. The bearing jaws 287′ face in the proximal direction and are provided coaxially with the pivot axis 216′ with a bore 283′ through which the pin-shaped bearing shaft 284′ can be inserted in order to mount the tool element 218′ of the end effector part 220 for pivotal movement on a shaft. Somewhat distally of a connecting area 236′ between the webs 294′ and the pull section 232′, the pull section 232′ is provided with a through-opening 238′ which extends through the pull section 232′ in approximately parallelepipedal shape symmetrically with the plane of symmetry and transversely to the longitudinal axis 224′ and opens up an end face 240′ of the pull section 232′ which is slightly concavely curved and faces in the distal direction. The gap 234′ and the through-opening 238′ are connected to each other. A tool end 290′ facing in the distal direction projects from the pull section 232′, more specifically, such that a virtual extension thereof in the proximal direction would be diametrically opposed to the webs 294′. A clamping surface 244′ facing towards the longitudinal axis 224′ is provided with a transverse toothing 246′ which may have a height in the range of between 20 μm and 100 μm.

Serving to connect the end effector part 220′ to the end effector part 222′ is a connecting device 248′ with a first connecting element 252′, which is formed on the force transmitting section 249′, more specifically, in the form of a parallelepipedal recess which is open in a radial direction facing away from the longitudinal axis 224′. From a bottom 253′ of the recess there projects an elongated parallelepipedal strip 254′ which in a direction parallel to the longitudinal axis 224′ is somewhat shorter than the recess, but ends flush with the end face 226′. A width transverse to the longitudinal axis 224′ of the connecting element 252′ corresponds to a width of the through-opening 238′ and of the gap 234′.

The connecting device 248′ comprises a second connecting element 256′ which is of such shape that it fills out the recess defined by the connecting element 252′ completely. It is therefore overall of approximately parallelepipedal shape and has an outer surface section which complements the force transmitting section 249′ to form a cylinder. To accommodate the strip 254′ there is formed in the connecting element 256′ a groove 258′ which is closed proximally. The strip 254′ can therefore engage the groove 258′, but a movement of the two connecting elements 252′ and 256′ relative to each other parallel to the longitudinal axis 224′ is prevented.

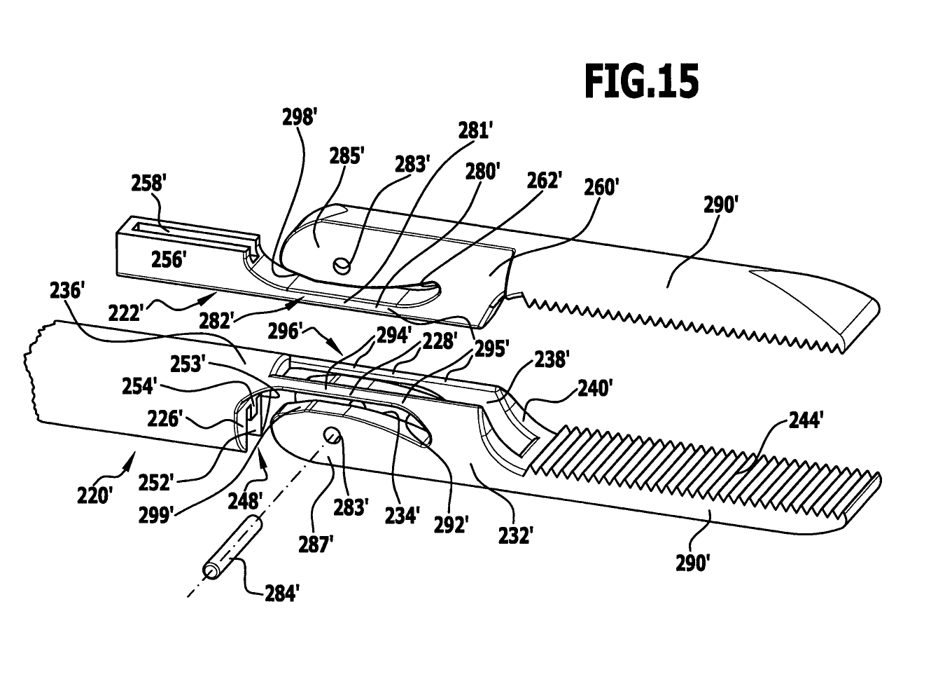

Diametrically opposed to the webs 294′, there extends in the distal direction from the connecting element 256′ a web 280′ defining an articulation member 281′, which forms a flexure hinge 282′ for transmitting push-and-pull forces from the force transmitting section 249′ onto the tool element 218′ comprised by the second end effector part 222′. The web 280′ ends at a jaw-shaped pull section 260′, from which there projects in the proximal direction a bearing jaw 285′, obliquely facing the longitudinal axis 224′, which has a bore 283′. The bearing jaw 285′ is separated from the web 280′ by a short gap 262′ extending in the distal direction somewhat beyond the pivot axis 216′. The pull section 260′, the web 280′ and the bearing jaw 285′ are all of identical width, and their width corresponds to a width of the connecting element 256′ and therefore to a width of the through-opening 238′. This makes it possible, for assembly of the end effector 214′, to assemble the separately manufactured end effector parts 220′ and 222′, as shown in FIG. 15, in such a way that by inserting the connecting element 256′ into the through-opening 238′ from above, i.e., facing the clamping surface 244′ and the longitudinal axis 224′, the connecting element 256′ can be pushed through the through-opening 238′ until it projects on the other side. The end effector parts 220′ and 222′ are then pivoted relative to each other in such a way that the connecting element 256′ can engage the connecting element 252′. The bores 283′ and 288′ are then in alignment, and a tool end 290′ extending in the distal direction from the pull section 260′, which then assumes a normal position as shown in FIGS. 13 and 14, is arranged symmetrically and opposite the other tool end 290′. The end effector 214′ can then be fixed with the bearing shaft 284′ on a shaft of the instrument. Optionally, the end effector parts 220′ and 222′ can be adhesively bonded and/or welded in the area of the connecting device 248′. If the force transmitting section 249′ is guided in a sleeve-shaped shaft, this prevents separation of the two parts, so that adhesive bonding or welding is not absolutely necessary.

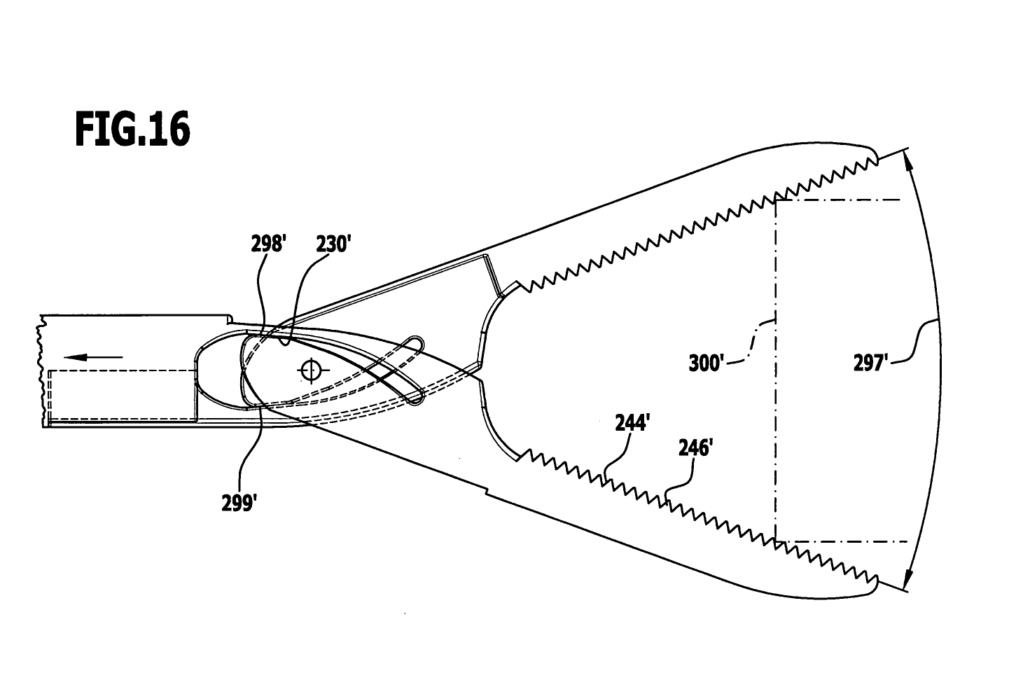

Owing to the penetrating or engaging arrangement of the tool elements 218′, the end effector 214′ is constructed such that a pushing force introduced by a force transmitting member results in an opening movement of the end effector 214′, i.e., the clamping surfaces 244′ are pivoted away from each other. The articulation members 281′ and 295′ are thereby bent in the direction towards the pivot axis 216′. From a certain opening angle 297′ of the tool elements 218′ onward, the bearing jaws 285′ and 287′ can touch the articulation members 281′ and 295′. There are formed on the bearing jaws 285′ and 287′ in the area of the proximal ends thereof small, flat articulation member supporting sections 298′ and 299′ of quadrilateral configuration, which define a surface area, and which in the normal position shown in FIGS. 13 and 14 essentially face the articulation members 281′ and 295′, respectively, but are spaced from these. If a certain opening angle 297′ is exceeded, the articulation member supporting sections 298′ and 299′ lie with surface-to-surface contact against the articulation members 281′ and 295′, respectively, as shown by way of example in FIG. 16. If the two tool elements 218′ are blocked against each other, for example, by an article 300′ held clamped between the clamping surfaces 244′, which may be, for example, tissue or part of an instrument, the tool elements 218′ are unable to move further towards each other, even when a pulling force is applied to the force transmitting section 249′. The introduction of pulling forces would, however, result in a so-called “tautening” of the flexure hinges 282′ and 296′, as a result of which high notch tensions could occur in the initial and end areas of the flexure hinges 282′ and 296′. Such notch tensions and also component loading in the loaded state of the tool elements 218′ at large opening angles 297′, as shown, for example, in FIG. 16, can be reduced by the supporting of the articulation members 281′ and 295′ by means of the articulation member supporting sections 298′ and 299′ of the bearing jaws 285′ and 287′. Furthermore, the stiffness of the end effector 214′ during gripping operations is increased by the supporting. The described supporting effect already occurs, in dependence upon the respective load situation, at opening angles 297′ of the tool elements 218′ below a maximum target opening angle, i.e., supporting effects, as described, are to be observed at the flexure hinges 282′ and 296′ during gripping operations at large opening angles 297′. The special supporting characteristics are achieved, in particular, by the articulation member supporting sections 298′ and 299′ being formed proximally of the pivot axis 216′.

The end effectors 14, 214, 214′ and 314, in particular, their end effector parts 102, 220 and 222, 220′ and 222′ and 320 and 322 may be made of a metal, in particular, an instrument steel, or of a plastic material. In particular, machining operations, such as grinding, milling, wire eroding or laser cutting are suited for metals. In particular, thermal forming processes such as, for example, deep drawing, hot blowing, stamping or injection molding may be used for processing plastic materials. In particular, polyetheretherketone (PEEK), polyethylene (PE), polyurethane (PU), polyethylene terephthalate (PET), polycarbonate (PC) or polypropylene (PP) are suited as plastic materials. In particular, end effectors made of plastic materials enable inexpensive manufacture of the shaft and the end effector together as one-way component which can be disposed of after a surgical procedure. In particular, owing to the small dimensions that are possible in the described end effectors, with, in particular, outer diameters of less then 2 mm being possible, a recycling of the end effectors is not easy and possibly involves the risk that sterility cannot be guaranteed.

The described end effectors are merely examples of end effectors comprising tool elements which are mounted for pivotal movement on a bearing shaft and to which a pushing and/or pulling force can be applied by a force transmitting member through a flexible articulation member in the form of a flexure hinge.

A resetting from a swivelled position of the tool elements back into a normal position in which the respective flexure hinges 96, 282, 296, 282′, 296′ and 396 are undeformed can be carried out by means of a resetting device 164, 264, 264′ and 364, respectively. This comprises a resetting member in the form of the flexible webs 94, 280 and 294, 280′ and 294′ and 380 and 394, respectively. Energy stored as a result of a deformation of the webs can be used to return the tool elements to their normal position. Therefore, the described end effectors may optionally also be used in such a way that only pulling or pushing forces need ever be introduced for actuation thereof.

1. Surgical instrument having a shaft, an end effector comprising at least one movably mounted tool element and defining a longitudinal axis, and a force transmitting member mounted in the shaft for movement in the distal and proximal directions for transmitting an actuating force introduced at a proximal end of the instrument onto the end effector in order to move the at least one tool element from a first tool element position to a second tool element position and/or vice versa, the at least one tool element being pivotably mounted on a bearing shaft held on the shaft, the at least one tool element being connected by at least one articulation member, at least a section of which is flexible, to the force transmitting member in order to transmit the actuating force from the force transmitting member onto the at least one tool element.

2. Surgical instrument in accordance with claim 1, wherein the at least one articulation member is undetachably connected to the at least one tool element.

3. Surgical instrument in accordance with claim 1, wherein the at least one articulation member is formed in one piece with the at least one tool element.

4. Surgical instrument in accordance with claim 1, wherein the at least one articulation member is undetachably connected to the force transmitting member.

5. Surgical instrument in accordance with claim 1, wherein the at least one articulation member is formed in one piece with the force transmitting member.

6. Surgical instrument in accordance with claim 1, wherein the at least one articulation member forms a flexure hinge between the force transmitting member and the at least one tool element.

7-8. (canceled)

9. Surgical instrument in accordance with claim 1, wherein the at least one articulation member engages the at least one tool element distally of the bearing shaft.

10-11. (canceled)

12. Surgical instrument in accordance with claim 1, wherein the end effector comprises a force transmitting section movable in the distal and proximal directions, which is connected to or lies against the force transmitting member and forms a distal end area of the force transmitting member, and wherein a proximal end of the at least one articulation member is connected to the force transmitting section.

13. Surgical instrument in accordance with claim 12, wherein the at least one articulation member is formed in one piece with the force transmitting section.

14. (canceled)

15. Surgical instrument in accordance with claim 1, wherein the force transmitting member comprises at least one piston mounted for displacement in the shaft, and a fluid section separated from the at least one piston by a sealing element and filled with a fluid which is pressable against the sealing element by the application of pressure in order to move the at least one piston lying against the sealing element parallel to the longitudinal axis of the shaft.

16-20. (canceled)

21. Surgical instrument in accordance with claim 1, comprising a guide device for guiding a movement of at least one of the force transmitting section and the force transmitting member in the shaft parallel to the longitudinal axis.

22-30. (canceled)

31. Surgical instrument in accordance with claim 1, wherein the at least one articulation member is deformed in any operating position of the at least one tool element that differs from a normal position in which it is undeformed.

32. Surgical instrument in accordance with claim 1, wherein the at least one articulation member extends parallel or substantially parallel to the longitudinal axis both proximally and distally of the bearing shaft.

33-34. (canceled)

35. Surgical instrument in accordance with claim 1, wherein the articulation member has a thickness ranging from approximately 50 μm to 700 μm.

36. Surgical instrument in accordance with claim 1, wherein a resetting device is provided for transferring the at least one tool element from any swivelled position of the tool element back into a normal position.

37-42. (canceled)

43. Surgical instrument in accordance with claim 1, wherein the end effector is of two-part construction and comprises two end effector parts.

44-45. (canceled)

46. Surgical instrument in accordance with claim 43, wherein the end effector is formed by two identical end effector parts fixedly connected to each other, which are arranged in a manner rotated through 180° about the longitudinal axis relative to each other.

47. Surgical instrument in accordance with claim 1, wherein the two tool elements are constructed so as to mutually penetrate or intersect each other.

48-53. (canceled)

54. Surgical instrument in accordance with claim 1, wherein the end effector is so constructed that as a result of a movement of the force transmitting member in the proximal direction, the at least one tool element is pivotable towards the longitudinal axis and vice versa.

55. Surgical instrument in accordance with claim 1, wherein the end effector is so constructed that as a result of a movement of the force transmitting member in the distal direction, the at least one tool element is pivotable towards the longitudinal axis and vice versa.

56-61. (canceled)

62. Surgical instrument in accordance with claim 1, wherein an articulation member supporting section facing the at least one articulation member is formed at a proximal end of the at least one tool element.

63-64. (canceled)

65. Surgical instrument in accordance with claim 62, wherein in a position of the tool element defining a pulled position, the articulation member supporting section is supported with surface-to-surface contact on the at least one articulation member, and, in the pulled position, the at least one tool element is swivelled out and blocked in the swivelled-out position.

66-74. (canceled)
